(19) United States
(12) Patent Application Publication
(10) Pub. No.: US 2017/0049560 A1
(43) Pub. Date: Feb. 23, 2017

(54) Intraocular Lens Holder

(71) Applicant: SCOTT A. CHERNE, ALVARADO, TX (US)

(72) Inventor: SCOTT A. CHERNE, ALVARADO, TX (US)

(21) Appl. No.: 14/831,984

(22) Filed: Aug. 21, 2015

(51) Int. Cl. A61F 2/16

(52) U.S. Cl. CPC A61F 2/16; A61F 2002/169; A61F 2002/16902

(57) ABSTRACT

A system and method for retaining an intraocular lens in an annular pouch implanted into a capsular bag of an eye. The annular pouch uses a circular ridge to maintain placement of the lens relative to the eye. Clarity of the capsular bag is retained over time because the pouch fills the capsular bag and prevents a loss of clarity in the capsular bag due to sagging.

### CROSS REFERENCE TO RELATED APPLICATIONS

This application shares technical disclosure in common with PCT Application No. PCT/US15/45920, filed 19 Aug. 2015, titled “INTRAOCULAR LENS HOLDER,” all of which is hereby incorporated by reference for all purposes as if fully set forth herein.

### BACKGROUND

1. Field of the Invention

The present invention relates generally to holders for intraocular lenses, and more specifically to a system and method for retaining the shape of a capsular bag with an intraocular lens holder.

2. Description of Related Art

During a cataract surgery the patient's cataracts are removed and replaced with a new lens. Typically the lens is retained merely by the capsular bag of the patient's eye. Capsular bags are extremely fragile and may not effectively secure lenses. Unsecured lenses cause vision problems due to misalignment. Furthermore, the capsular bag over time wrinkles causing vision problems and contraction of the capsular bag over the struts of the replacement lens prevents the replacement lens from being removed from the eye in the future. Currently, there are a limited few lenses that have eyelets in their haptics that can be sewn to the eye wall to stabilize them but they often tilt causing visual distortions and once they are sewn in no new lenses can be placed, nor can they be exchanged should the need arise without a major eye surgery. Thus, there exists significant room for improvement in the art for overcoming these and other shortcomings of conventional systems and methods for securing intraocular lenses.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1A is a plan diagram of an intraocular lens pouch according to the present application;

FIG. 1B is a cross section diagram of an intraocular lens pouch from FIG. 1a taken at line Ib-Ib according to the present application;

FIG. 2A is a plan diagram of an intraocular lens pouch with two lenses according to the present application;

FIG. 2B is a cross section diagram of the intraocular lens pouch of FIG. 2a taken at line IIb-IIb according to the present application;

FIG. 3 is a cross section diagram of an intraocular lens pouch in an eye according to the present application;

FIG. 4 is a plan diagram of an intraocular lens pouch with eyelets according to the present application;

FIG. 5 is a cross section diagram of an intraocular lens pouch with eyelets according to the present application;

FIG. 6 is a plan diagram of an alternative intraocular lens pouch according to the present application;

FIG. 7A is a plan diagram of a cloverleaf shaped intraocular lens pouch according to the present application; and

FIG. 7B is a cross section diagram of the cloverleaf intraocular lens pouch from FIG. 7 taken at line VIIb-VIIb according to the present application.

While the assembly and method of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the intraocular lens pouch system and method are provided below. It will of course be appreciated that in the development of any actual embodiment, numerous implementation-specific decisions will be made to achieve the developer's specific goals, such as compliance with assembly-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

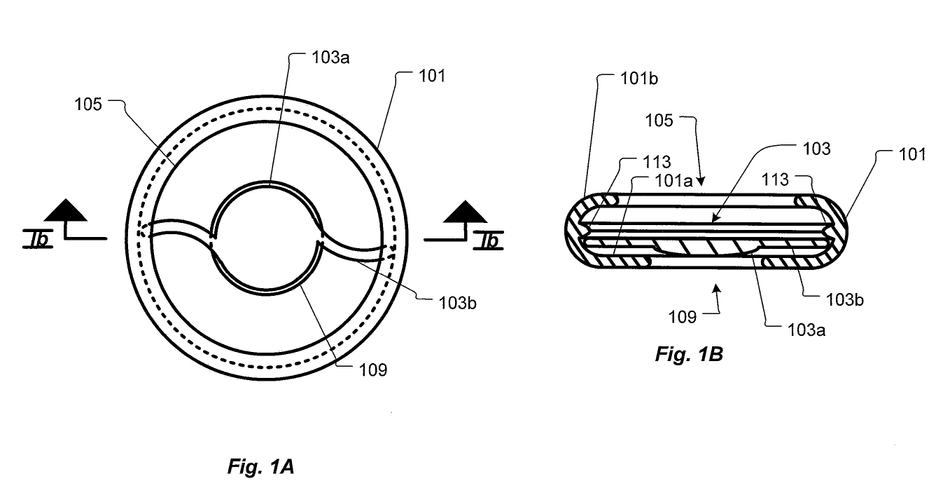

Referring now to FIGS. 1A and 1B in the drawings, a preferred embodiment of an intraocular lens pouch according to the present application is illustrated. Pouch 101 or ring is annular and or jelly bean shaped and configured for both retaining an intraocular lens and providing strength to the capsular bag of a user's eye. Jelly bean shaped should be understood to describe having a cross-sectional shape of a bean, where a shape of a top half of the pouch is different than a shape of a bottom half of the pouch. Pouch 101 has an inner surface 101a and an outer surface 101b. Intraocular lens 103 has a lens portion 103a and at least one haptic strut 103b. While the intraocular lens shown features two struts 103b it should be apparent that other variations of lenses are contemplated by this application such as lens with three and four struts. Pouch 101 entirely retains the lens 103 and acts as a barrier between the lens and the capsular bag. The lens 103 is not in contact with the eye and can easily be replaced because the eye will not have grown over the lens.

Pouch 101 is fabricated from high grade surgical silicone or acrylic suitable for implanting into an eye of a patient. Other materials are contemplated so long as they are suitable for surgically implanting into an eye. Pouch 101 has a first opening 105 and a second opening 109. First opening 105 is the larger of the two openings and has a diameter approximately 80% an outer diameter of the pouch 101. Second opening 109 is approximately the size of the lens portion 103a of the intraocular lens 103.

Pouch 101 has a ridge 113 that is inwardly facing and runs along the entire circumference of the pouch 101. Ridge 113 is located approximately half way between the first opening 105 and the second opening 109 and thereby creates a first notch and a second notch. Ridge divides the inner surface 101a of the pouch 101 into an upper inner surface and a lower inner surface. Intraocular lens 103 is retained by the haptic struts 103b of the lens being held between the ridge 113 and the inner surface 101a. As shown the lens 103 is located between the ridge 113 and the lower inner surface. However, the lens 103 can be located between the ridge 113 and the upper inner surface. The ridge 113 keeps the lens 103 parallel or generally parallel to the pouch and minimizes lens tilt and misplacement.

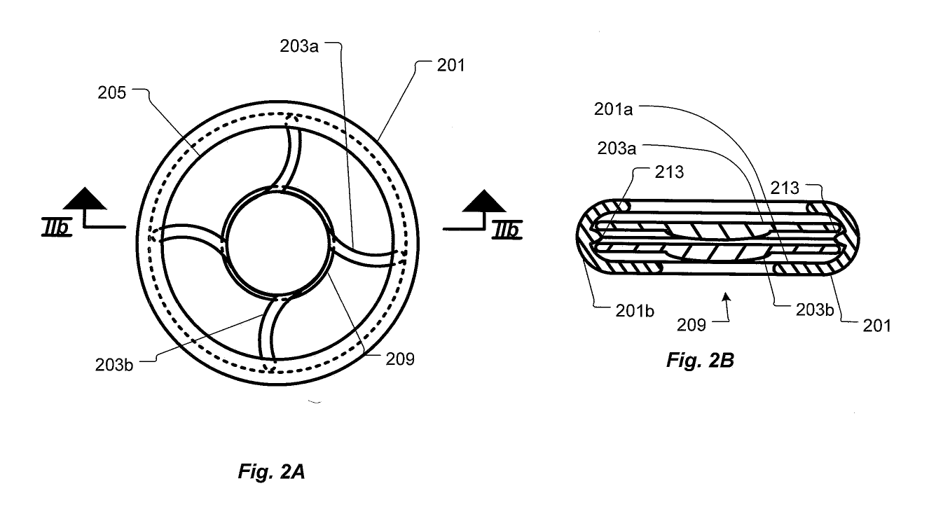

Referring now to FIGS. 2A and 2B in the drawings, an alternative embodiment of an intraocular lens pouch with dual intraocular lenses according to the present application is illustrated. Pouch 201 is similar to pouch 101, however pouch 201 holds two separate intraocular lenses.

Pouch 201 or holder is annular and or jelly bean shaped and configured for both retaining an intraocular lens and providing strength to the capsular bag of a user's eye. Pouch 201 has an inner surface 201a and an outer surface 201b. Intraocular lens 203 has a lens portion and at least one haptic strut. Pouch 201 is fabricated from high-grade surgical silicone, acrylic, or other materials suitable for implanting into an eye of a patient.

Pouch 201 has at least a first opening 205 and preferably a second opening 209. First opening 205 is the larger of the two openings and has a diameter approximately 80% an outer diameter of the pouch 201. Second opening 209 is approximately the size of the lens portion of the intraocular lens 203.

Pouch 201 has a ridge 213 that is inwardly facing and runs along the entire circumference of the pouch 201. Ridge 213 is located approximately half way between the first opening 205 and the second opening 209. Ridge divides the inner surface 201a of the pouch 201 into an upper inner surface and a lower inner surface. First intraocular lens 203a is retained by the haptic struts of the lens being held between the ridge 213 and the inner surface 201a. As shown the first lens 203a is located between the ridge 213 and the lower inner surface and the second lens 203b is located between the ridge 213 and the upper inner surface.

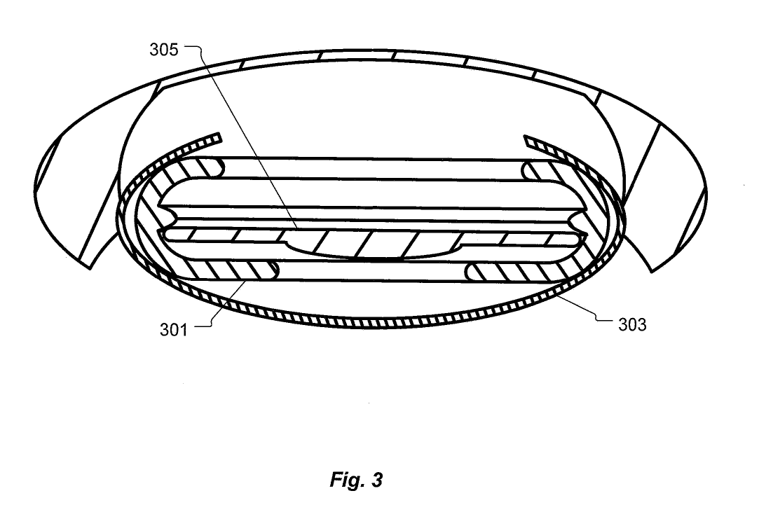

Referring now to FIG. 3 in the drawings, an alternative embodiment of an intraocular lens pouch in an eye according to the present application is illustrated. Pouch 301 is surgically implanted into capsular bag 303 of a user during a cataract or crystalline lens removal. To implant the pouch 301 an incision is made into an anterior side of the capsular bag 303 and the pouch 301 is implanted. Next the intraocular lens 305 is inserted into the pouch through the incision. The haptic struts or legs of the lens 305 are expanded such that the lens is retained between the ridge of the pouch and the pouch itself. The incision is closed and the pouch 301 and lens 305 is held securely in the capsular bag 303 of the patient.

Lens 305 can be readily replaced or rotated by eye surgery because the eye will not have grown over the haptic struts of the lens 305. Therefore as a patient's eye changes over time, or as newer and better lenses become available, the lens can be readily changed without extensive damage to the eye from cutting out the previous lens. Furthermore, because the pouch 301 is shaped like a flattened sphere the capsular bag retains a filled shape and remains more optically clear.

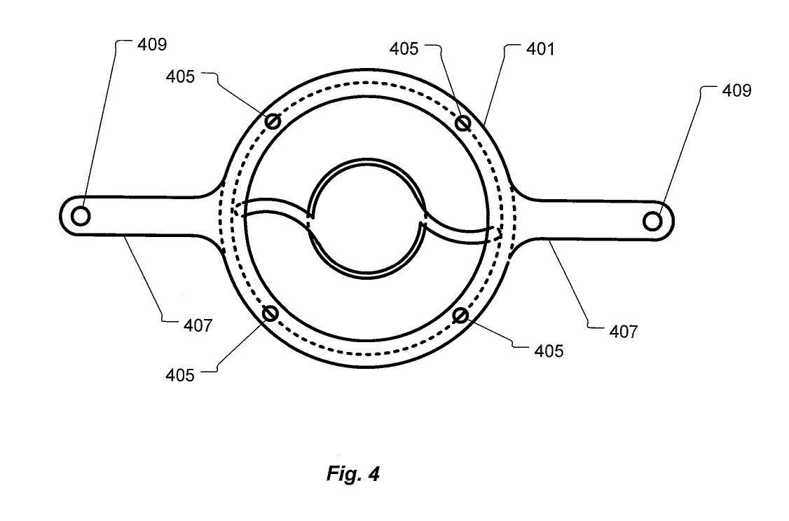

Referring now also to FIG. 4 in the drawings, an alternative embodiment of an intraocular lens pouch according to the present application is illustrated. Pouch 401 is comprised of elements like pouch 101 however pouch 401 further comprises at least one hole 405, arms 407, and eyelets 409. Eyelets 409 are configured to allow a surgeon to suture the pouch to the scleral wall of the eye. Arms 407 protrude from the pouch and are configured to place the eyelets 409 exterior to the capsular bag when the pouch is installed in an eye. Eyelets 409 are opening through the arms 407 suitable for suturing the pouch 401 into an inner scleral wall of an eye.

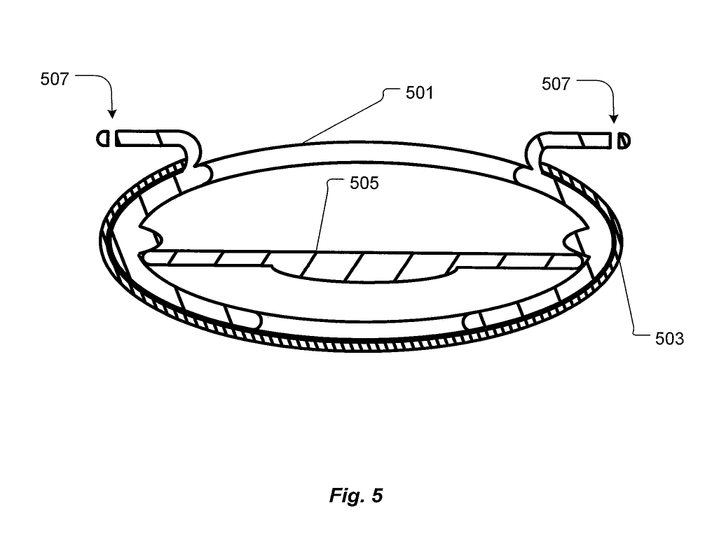

Referring now to FIG. 5 in the drawings, an alternative embodiment of an intraocular lens pouch with eyelets in a capsular bag according to the present application is illustrated. Pouch 501 is surgically implanted into capsular bag 503 of a user during a cataract or crystalline lens removal. To implant the pouch 501 an incision is made into an anterior side of the capsular bag 503 and the pouch 501 is implanted. Next the intraocular lens 505 is inserted into the pouch through the incision. The haptic struts or legs of the lens 505 are expanded such that the lens is retained between the ridge of the pouch and the pouch itself. The incision is closed and the pouch 501 and lens 505 is held securely in the capsular bag 503 of the patient. Pouch 501 is further comprised of a first arm and a second arm each containing an eyelet 507 located through the arm. Eyelets 507 are configured to allow a surgeon to suture the pouch to the scleral wall of the eye.

Lens 505 can be readily replaced or rotated by eye surgery because the eye will not have grown over the haptic struts of the lens 505. Therefore as a patient's eye changes over time, or as newer and better lenses become available, the lens can be readily changed without extensive damage to the eye from cutting out the previous lens. Furthermore, because the pouch 501 is shaped like a jelly bean the capsular bag retains a jelly bean shape and remains more optically clear.

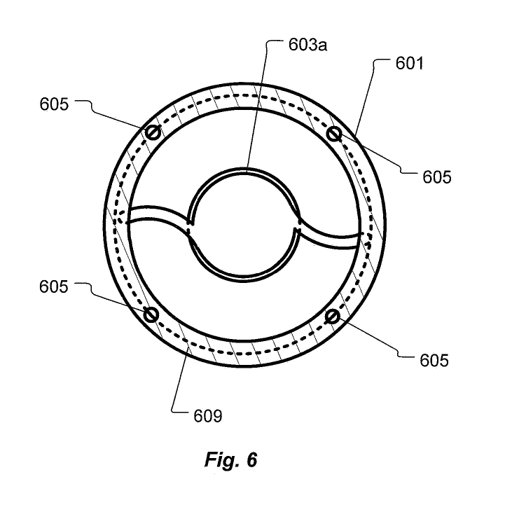

Referring now also to FIG. 6 in the drawings, an alternative embodiment of an intraocular lens pouch according to the present application is illustrated. Pouch 601 is comprised of elements like pouch 101 however pouch 601 further comprises at least one hole 605, and surface texturing 609. The at least one hole 605 is preferably four holes, equally spaced around the pouch, for use as anterior positioning holes. Hole 605 goes through a lip of the pouch and provides users assistance with manipulation of the pouch when in the eye. The user can adjust the position of the pouch relative to the eye by pushing and pulling on the pouch with the hole. The hole provides a better location to grip the pouch. Surface texturing 609 provides enhanced capsular grasp of the pouch 601. Surface texturing 609 is a series of slight ridges or grooves located entirely across the exterior surface of the pouch. Alternative embodiments of the surface texturing include pitting or other slight surface irregularities to enhance the capsular grasp of the pouch 601.

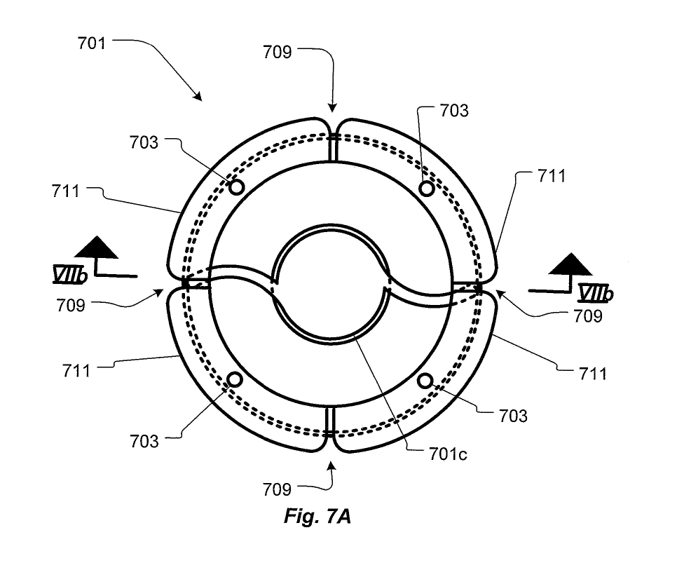

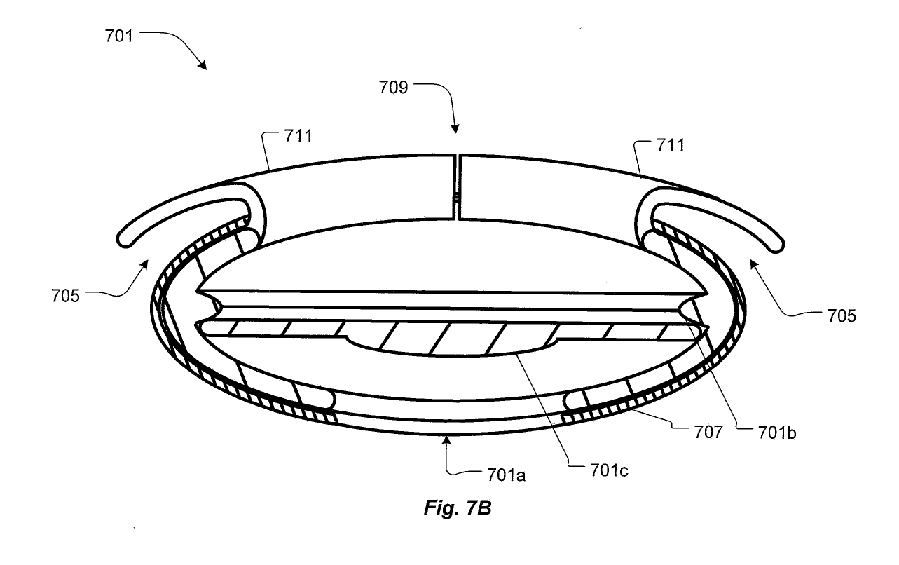

Broken or torn posterior lens capsule are improved with a pouch that can help provide strength and retain the broken or torn capsular bag together. Referring now to FIGS. 7A and 7B in the drawings, a preferred embodiment of a cloverleaf shaped intraocular lens pouch according to the present application is illustrated in a torn capsular bag. Pouch 701 is comprised of elements like pouch 101 such as an opening 701a and a ridge 701b to retain a lens 701c; however, pouch 701 further comprises four radially spaced holes 703 and the pouch flares outwardly creating an annular channel 705 around the entire exterior surface of the pouch to retain an edge of a broken capsular bag 707. Pouch 701 features notches 709 cut into the upper surface or channel 705 to shape the pouch into a cloverleaf shape having four petals 711. Notches provide flexibility to the pouch during insertion. While the pouch 701 is shown with four petals or extensions it should be apparent that pouches with more or less petals are contemplated by this application. The cloverleaf design enable placement of the petals 711 to overlap the anterior lens capsule so the pouch 701 remains centered and in anatomical position. The anterior curvature of the pouch 701 extends peripherally over the anterior capsular rim opening so that the pouch 701 does not drop into the back of an eye through the posterior capsular opening.

The pouch or holder is an improvement over the current technology because: it increases the strength and maintains the shape of the capsular bag by filling the bag; it allows a lens to be replaced, repositioned, or rotated later because the capsular bag doesn't grow over the struts; and because the capsular bag doesn't sag over time and retains its rounded shape, it is anticipated that the untorn bag remains clearer over time which reduces or eliminates the need to have a second laser eye surgery of a clouded bag.

It is apparent that a system with significant advantages has been described and illustrated. The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. An intraocular lens pouch, comprising:
a ring having;
a first opening;
a second opening;
a ridge located between the first opening and the second opening;
wherein the ring is configured to provide support for a capsular bag.

2. The intraocular lens pouch according to claim 1, wherein the first opening is larger in diameter than the second opening.

3. The intraocular lens pouch according to claim 1, wherein the ring is fabricated from surgical silicone.

4. The intraocular lens pouch according to claim 1, wherein the ring is fabricated from surgical acrylic

5. A pouch for securing an intraocular lens in a capsular bag, comprising:
a member with an annular shape having;
an upper inner surface;
a lower inner surface; and
a ridge located between the upper inner surface and the lower inner surface.

6. The pouch according to claim 5, wherein the intraocular lens is retained between the lower inner surface and the ridge.

7. The pouch according to claim 6, further comprising:
four holes through the member configured for anterior positioning.

8. A system for securing an intraocular lens in a capsular bag, comprising:
a pouch with a jelly bean shape having;
at least one opening;
a ridge located inside the opening;
wherein the ridge is configured for securing a first intraocular lens.

9. The system for securing an intraocular lens in a capsular bag according to claim 8, wherein the ridge retains the first intraocular lens generally parallel.

10. The system for securing an intraocular lens in a capsular bag according to claim 8, wherein the ridge is configured for securing a second intraocular lens.

11. The system for securing an intraocular lens in a capsular bag according to claim 10, wherein the ridge retains the second intraocular lens generally parallel to the first intraocular lens.

12. The system for securing an intraocular lens in a capsular bag according to claim 8, further comprising:
at least one hole configured for anterior positioning.

13. The system for securing an intraocular lens in a capsular bag according to claim 8, further comprising:
an arm;
at least one eyelet located in the arm configured for suturing.

14. The system for securing an intraocular lens in a capsular bag according to claim 13, wherein the arm is configured for locating the eyelet exterior to the capsular bag.

15. The system for securing an intraocular lens in a capsular bag according to claim 8, wherein an exterior surface of the pouch is textured.

16. The system for securing an intraocular lens in a capsular bag according to claim 15, wherein the texturing provides adhesion between the pouch and the capsular bag.

17. The system for securing an intraocular lens in a capsular bag according to claim 8, further comprising:
a channel located around an exterior of the pouch;
wherein the channel is configured for retaining an edge of the capsular bag.

18. The system for securing an intraocular lens in a capsular bag according to claim 17, wherein the channel is notched.

19. The system for securing an intraocular lens in a capsular bag according to claim 18, further comprising:
at least one extension for retaining an edge of the capsular bag.
